(19) United States
(12) Patent Application Publication
(10) Pub. No.: US 2025/0029733 A1
(43) Pub. Date: Jan. 23, 2025

(54) DETERMINING A GENETIC PREDISPOSITION OF A MEDICAL CAUSE

(71) Applicant: International Business Machines Corporation, Armonk, NY (US)

(72) Inventors: Ratika Mehta, Gurgaon (IN); Anita Duggal, Delhi (IN); Mukundan Sundararajan, Bangalore (IN); Sudarshan ., Ghaziabad (IN)

(21) Appl. No.: 18/356,344

(22) Filed: Jul. 21, 2023

(51) Int. Cl. G16H 50/70; G06F 40/35; G16H 10/20; G16H 50/20

(52) U.S. Cl. CPC G16H 50/70; G06F 40/35; G16H 10/20; G16H 50/20

(57) ABSTRACT

Embodiments of the present invention provide an approach for enabling medical professionals in determining a genetic predisposition of a medical cause from a family tree analysis. Specifically, the approach involves receiving medical symptoms of a patient and medical narrations about family members, which are organized in a family tree. The medical narrations are collected using questions generated using a supporting symptoms list. The narrations are filtered to provide a summary of relevant information. A linked tree of related words is established based on the summary. A natural language processing engine generates insights based on an analysis of the summary. Likelihood paths of genetic susceptibility to a medical condition through the family are determined by identifying family member symptoms in the linked tree of related words. These likelihood paths are then matched with a known inheritance pattern of a disease to provide an alert to the family members about the disease.

### TECHNICAL FIELD

The present invention relates to medical data collection and analysis, and more specifically to embodiments for determining a genetic predisposition of a medical cause from a family tree analysis.

### BACKGROUND

In cases where patients are suffering from genetic or systemic diseases that have not responded to symptomatic treatments and where known treatment protocols do not seem applicable, the field of genetic disease likelihood and treatment is rapidly evolving. In such situations, it becomes crucial for doctors to have access to the medical history of not only the affected individual but also the entire immediate or extended family. By understanding the genetic makeup and medical conditions of family members, healthcare professionals can gain valuable insights into the potential hereditary nature of the disease, enabling them to explore alternative treatment options and personalized approaches. This comprehensive understanding of the family medical history can guide doctors in making informed decisions about genetic testing, targeted therapies, and potential clinical trials, that can provide renewed hope and potential breakthroughs for patients facing complex and challenging conditions.

The importance of accessing the medical history of the entire or affected members of the immediate or extended family is magnified in the rapidly evolving field of genetic disease likelihood and treatment. Advances in genetic research and technology have opened up new possibilities for precision medicine and personalized treatments. By analyzing the genetic profiles and medical histories of family members, doctors can identify patterns, potential genetic mutations, or inherited conditions that may contribute to the patient's disease. This information can be invaluable in determining the most appropriate course of action, such as genetic counseling, targeted therapies, or experimental treatments. Access to comprehensive family medical history empowers healthcare professionals to tailor treatment plans specifically for the patient's unique genetic makeup, that can increase the chances of successful outcomes.

### SUMMARY

Embodiments of the present invention provide an approach for determining a genetic predisposition of a medical cause from a family tree analysis. Specifically, the approach involves receiving medical symptoms of a patient and medical narrations about family members, which are organized in a family tree. The medical narrations are collected using questions generated using a supporting symptoms list. The narrations are filtered to provide a summary of relevant information. A linked tree of related words is established based on the summary. A natural language processing engine generates insights based on an analysis of the summary. Likelihood paths of genetic susceptibility to a medical condition through the family are determined by identifying family member symptoms in the linked tree of related words. These likelihood paths are then matched with a known inheritance pattern of a disease to provide an alert to the family members about the disease.

A first aspect of the present invention provides a method for determining a genetic predisposition of a medical cause from a family tree analysis, comprising: receiving, by the natural language understanding engine, medical narrations from family members of the patient represented in a family tree by asking the family members the generated set of questions; filtering, by a cognitive system, the medical narrations to provide a summary; generating, by the natural language understanding engine, insights based on the summary using a natural language processing technique; establishing, by the cognitive system, a linked tree of related words based on the insights; determining, by the cognitive system, likelihood paths through the family based on symptoms identified in the linked tree of related words; and matching, by the cognitive system, the determined likelihood paths with a known inheritance pattern of a disease to provide an alert to the family members about the disease.

A second aspect of the present invention provides a computing system for determining a genetic predisposition of a medical cause from a family tree analysis, comprising: a processor; a memory device coupled to the processor; and a computer readable storage device coupled to the processor, wherein the storage device contains program code executable by the processor via the memory device to implement a method, the method comprising: receiving, by the natural language understanding engine, medical narrations from family members of the patient represented in a family tree by asking the family members the generated set of questions; filtering, by a cognitive system, the medical narrations to provide a summary; generating, by the natural language understanding engine, insights based on the summary using a natural language processing technique; establishing, by the cognitive system, a linked tree of related words based on the insights; determining, by the cognitive system, likelihood paths through the family based on symptoms identified in the linked tree of related words; and matching, by the cognitive system, the determined likelihood paths with a known inheritance pattern of a disease to provide an alert to the family members about the disease.

A third aspect of the present invention provides a computer program product for determining a genetic predisposition of a medical cause from a family tree analysis, the computer program product comprising a computer readable storage device, and program instructions stored on the computer readable storage device, to: receive, by a natural language understanding engine, medical symptoms of a patient; generate, by the natural language understanding engine, a set of questions from a supporting symptoms list; receive, by the natural language understanding engine, medical narrations from family members of the patient represented in a family tree by asking the family members the generated set of questions; filter, by a cognitive system, the medical narrations to provide a summary; generate, by the natural language understanding engine, insights based on the summary using a natural language processing technique; establish, by the cognitive system, a linked tree of related words based on the insights; determine, by the cognitive system, likelihood paths through the family based on symptoms identified in the linked tree of related words; and match, by the cognitive system, the determined likelihood paths with a known inheritance pattern of a disease to provide an alert to the family members about the disease.

### BRIEF DESCRIPTION OF THE DRAWINGS

FIG. 1 depicts a block diagram illustrating an example of an environment for the execution of at least some of the computer code involved in performing the inventive methods, in accordance with embodiments of the present invention.

FIG. 2 depicts a block diagram of a system architecture involved in performing the inventive methods, in accordance with embodiments of the present invention.

FIG. 3 depicts a flow diagram for determining a genetic predisposition of a medical cause from a family tree analysis, in accordance with embodiments of the present invention.

### DETAILED DESCRIPTION

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

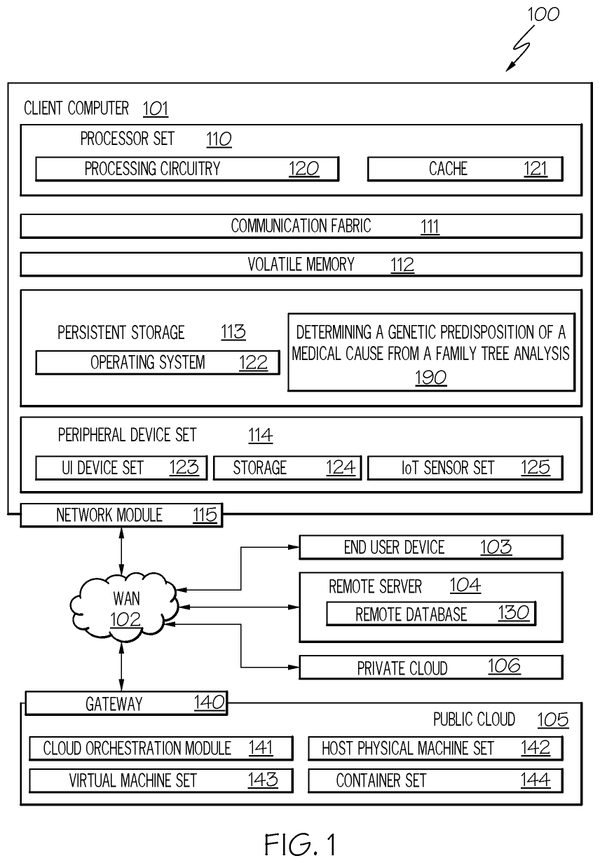

Computing environment 100 of FIG. 1 contains an example of an environment for the execution of at least some of the computer code involved in performing the inventive methods, such as determining a genetic predisposition of a medical cause from a family tree analysis technique 190. In addition to block 190, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 190, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 190 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface type operating systems that employ a kernel. The code included in block 190 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

In the present disclosure, use of the term “a,” “an”, or “the” is intended to include the plural forms as well, unless the context clearly indicates otherwise. Also, the term “includes,” “including,” “comprises,” “comprising,” “have,” or “having” when used in this disclosure specifies the presence of the stated elements, but do not preclude the presence or addition of other elements.

Embodiments of the present invention provide an approach for determining a genetic predisposition of a medical cause from a family tree analysis. Specifically, the approach involves receiving medical symptoms of a patient and medical narrations about family members, which are organized in a family tree. The medical narrations are collected using questions generated using a supporting symptoms list. The narrations are filtered to provide a summary of relevant information. A linked tree of related words is established based on the summary. A natural language processing engine generates insights based on an analysis of the summary. Likelihood paths of genetic susceptibility to a medical condition through the family are determined by identifying family member symptoms in the linked tree of related words. These likelihood paths are then matched with a known inheritance pattern of a disease to provide an alert to the family members about the disease.

In the context of genetic/systemic diseases where symptomatic treatments have been ineffective and known protocols are not applicable, the field of genetic disease likelihood and treatment is rapidly advancing. It can be crucial for doctors to have access to the medical history of the entire or affected members of the immediate or extended family. This information not only enhances doctors' understanding of the disease but also aids in predicting its progression. In cases where affected family members have passed away and no medical records are available, it becomes challenging for patients or caregivers to gather unbiased information about symptoms and treatments. Timely access to relevant medical documents can have life-altering implications in such situations.

As scientific and technological advancements continue, additional research is being conducted worldwide on various diseases, including genetic conditions. However, the responsibility often falls on patients or caregivers to navigate through the numerous clinical trials and determine their eligibility. Frequently, patients and caregivers struggle to fully comprehend the medical terminology and specific criteria required for participation. This cumbersome process sometimes results in missed opportunities to access life-saving drugs that could have otherwise been beneficial.

Patients with genetic diseases often need to consult doctors from different specialties, hospitals, and geographical locations. Each visit to a new doctor necessitates the patient to reiterate their entire medical history. This process is burdensome for both patients and doctors, as it requires reviewing numerous relevant documents and comprehending the complete medical background in a single session. Patients or caregivers who are not well-versed in all the details may struggle to advocate for themselves or may unintentionally omit crucial information that could aid in diagnosing the disease.

The deficiencies in current methods include incomplete patient record management and lack of analysis on familial relations when diagnosing diseases using decision support systems. Current methods typically only analyze patients' self-reported symptoms and don't adequately consider heritable factors present within families leading to suboptimal treatment plans and resource allocation. The approach described herein creates a more accurate representation of health issues by supporting and mapping symptoms and relationships based on the occurrence within immediate and extended family members, considering the genetic origins of the disease known through the family tree.

Advantages of the described approach include increased efficiency, enhanced analysis and genetic identification, and transferable knowledge. By utilizing the natural language analytic and understanding engine, the approach can reduce the time and effort required for patients and medical professionals to determine their eligibility for clinical trials. It can more quickly match patient symptoms with ongoing trials, streamlining the process and facilitating access to potentially beneficial studies. The approach captures and represents the family's symptom history, which can allow for improved analysis and identification of genetic trails. This information can be used to make the larger family aware of potential genetic risks, enabling proactive measures for future medical conditions. This can help in identifying causes and developing targeted treatment protocols more efficiently. Further, the pathway predictors and learning from the approach can be fed into other individualized artificial intelligence (AI) systems. This transfer of knowledge can enhance the capabilities of these systems for unique instances, benefiting other patients by improving the accuracy and effectiveness of their diagnoses and treatment plans. This promotes continuous learning and advancement in medical AI technologies.

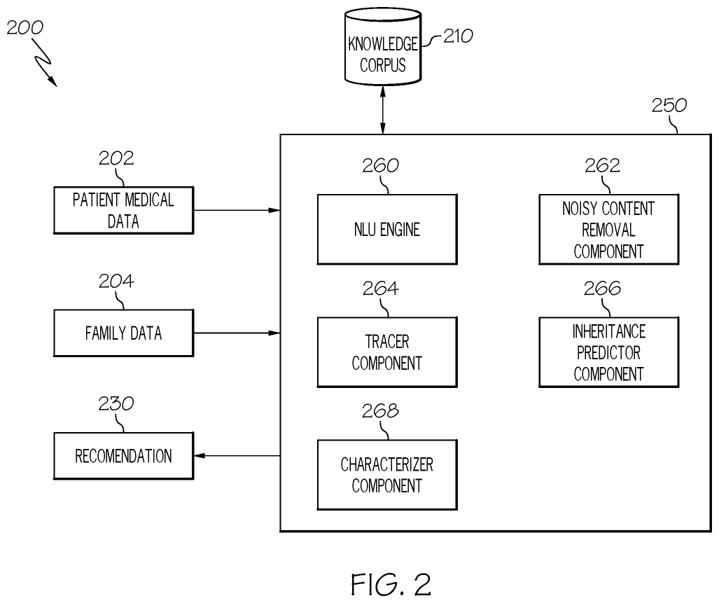

FIG. 2 depicts a block diagram of one or more components of a system environment 200 by which services provided by a medical data collection and analysis system (MDCA) 250, in accordance with an embodiment of the present disclosure. In accordance with some embodiments, the MDCA system 250 is configured to determine a genetic predisposition of a medical cause from a family tree analysis. The core component of the MDCA system 250 is a natural language processing/natural language understanding (NLP/NLU) engine 260. NLU is branch of natural language processing (NLP), which helps computers understand and interpret human language by breaking down the elemental pieces of speech. While speech recognition captures spoken language in real-time, transcribes it, and returns text, NLU goes beyond recognition to address a user's intent. For simplicity, the NLP/NLU engine 260 will be referred to as an NLU engine 260, as NLU is a branch of NLP that focuses on understanding and interpreting human language. Other functions not performed by NLU engine 260 can be described generically as being performed by a cognitive system. As used herein, the “cognitive system” refers to a computer-based system that is designed to mimic or simulate human cognitive processes, such as perception, learning, reasoning, and problem-solving. It typically involves the use of artificial intelligence and machine learning techniques to enable computers to understand and interpret data, make informed decisions, and perform tasks that require human-like cognitive abilities.

As used herein, an “engine” can refer to a hardware processing circuit, which can include any or some combination of a microprocessor, a core of a multi-core microprocessor, a microcontroller, a programmable integrated circuit, a programmable gate array, a digital signal processor, or another hardware processing circuit. Alternatively, an “engine” can refer to a combination of a hardware processing circuit and machine-readable instructions (software and/or firmware) executable on the hardware processing circuit.

Typically, an NLU engine 260 can be trained and used through a series of steps. The process typically begins with data collection, where a large dataset of text or speech data is gathered to represent the target language or domain comprehensively. This dataset covers various language patterns, topics, and contexts to ensure the engine's effectiveness. Once the dataset is collected, it undergoes preprocessing, which can involve cleaning the data by removing irrelevant information, correcting errors, and normalizing the text. It also includes linguistic tasks like tokenization, stemming, or lemmatization to prepare the data for further analysis. To train the NLU engine 260, the dataset can be annotated and labeled. Human annotators review the data and mark specific elements of interest, such as named entities, syntactic structures, semantic roles, sentiment, or intent. These annotations serve as the ground truth for training the engine's algorithms.

The next step is feature extraction, where relevant features are extracted from the annotated data. This can involve transforming the data into numerical or categorical representations that can be used as input for machine learning algorithms. Techniques like bag-of-words representations, word embeddings, or advanced methods like attention mechanisms or transformers are employed for feature extraction. With the extracted features, the NLU engine's 260 model can be trained using machine learning algorithms. Typically, supervised learning is used, where the model learns from the annotated data to map input text or speech to desired outputs. Various algorithms like support vector machines, random forests, or deep learning models such as recurrent neural networks or transformers can be utilized for training.

After training, the model can be evaluated using a separate set of annotated data called a validation set. Performance metrics like accuracy, precision, recall, and/or F1 score are calculated to assess the model's effectiveness. If the performance is not satisfactory, iterations of fine-tuning, adjusting hyperparameters, or updating the training data are performed until the desired performance is achieved. Once the model is trained and evaluated, it can be deployed for real-world applications. The NLU 260 engine can take input text or speech, apply preprocessing steps, and use the trained model to extract information, understand user intents, or generate appropriate responses. This enables the engine to power chatbots, virtual assistants, sentiment analysis systems, and other applications that require natural language processing. It's important to note that the training and usage of NLU engine 260 can require continuous monitoring, updating, and retraining to adapt to evolving language patterns, new vocabulary, or changing user needs. This ensures the engine remains effective and accurate over time.

At the start of an initial session, a medical professional or practitioner (e.g., a doctor) can retrieve and view a patient's electronic medical record, featuring the patient's medical history accumulated from past encounters (e.g., allergies, medications, immunization dates, laboratory and imaging results, vital signs, progress notes, etc.) and any available genetic profile via a family tree. If no prior records exist, the medical professional initiates documentation at that time. For identification purposes, every patient can receive a unique identifier (e.g., Social Security Number, etc.). Creating a family tree during this session is optional. As follow-ups occur, updates reflecting additional diagnoses, examinations, and test findings get added to the patient's medical data.

During the session, the medical professional can also collect the current symptoms of the patient from the patient and/or caregiver. A caregiver is typically a person who provides emotional, physical, or cognitive support to another individual who requires assistance due to illness, age, disability, or other reasons. The caregiver might be a family member, friend, paid employee, volunteer, or professional working within organizations offering supportive care. Patient symptoms can include any physical or subjective signs that a patient experiences and reports, indicating the presence of an illness, condition, or disease. Symptoms can manifest in various ways, such as pain, discomfort, abnormal sensations, changes in bodily functions, or alterations in behavior.

If there is reason to suspect a genetic condition, the medical professional can ask the patient (or caregiver) to provide a family tree and family medical information to understand potential genetic influences on the patient's health. The family tree might begin with the patient's immediate family members (parents, siblings, and children) and extend to grandparents, aunts, uncles, cousins, and other blood relatives if possible. Obtaining medical history data from family members in a family tree can be essential when trying to understand a person's risk for certain diseases and health conditions. This can include information regarding chronic conditions, major illnesses, diseases that tend to occur at a young age, unusual or severe complications, and/or diseases that affected several members of the family. Details regarding these conditions can include dates of diagnoses, ages at onset, signs and symptoms experienced, results of tests performed, medications prescribed, relevant medical history and management, and response to therapy. The patient and/or caregiver can work with extended relatives of the patient to compile any additional information and necessary documentation. To assist, the medical professional may provide a questionnaire for each member of the family tree to help gather the family medical history of those represented in the family tree.

In an embodiment, the questionnaire is designed using Natural Language Processing and Machine Learning (NLP/ML) technology to understand any pattern of disease inheritance. The design of a questionnaire using Natural Language Processing (NLP) and Machine Learning (ML) technology can involve several key steps. First, a diverse dataset can be collected, including information on disease inheritance patterns, symptoms, and language usage within families or tribes. The data is then preprocessed to clean and normalize it. Relevant features, such as symptom phrases and language indicators, can be extracted. NLP and ML algorithms can be selected and trained on the preprocessed data to learn patterns and relationships. The trained models can be validated and fine-tuned for improved performance. The NLP/ML models can be integrated into the questionnaire system, allowing users to input responses and receive relevant feedback based on the analyzed data. The models can be continuously updated and refined, ensuring the questionnaire adapts to changing patterns and improves its understanding of disease inheritance within diverse linguistic and cultural contexts.

The NLP/ML technology used in designing the questionnaire serves several purposes. It enables the system to process and analyze human language in multiple languages, allowing the questionnaire to be accessible to individuals who speak different languages within the family or tribe. In a medical context, the terms “family” and “tribe” can be used to refer to different aspects of patient care and genetic relationships. The technology utilizes machine learning algorithms to learn from patterns and data, enabling the questionnaire to include likely symptom phrases used within the family or tribe. This helps in understanding the specific language and terminology used when describing symptoms related to the disease. By leveraging NLP/ML technology, the questionnaire becomes more effective in capturing relevant information and understanding the pattern of disease inheritance within the family or tribe.

In addition, a supporting symptoms list can be used to generate questions to be asked to obtain additional facts from the blood relations of the patient. The supporting symptoms list represents a compilation of additional symptoms that often accompany a particular medical condition or ailment. These symptoms are considered supportive because they contribute to the overall clinical picture and can assist in making a more accurate diagnosis. This feature is designed to gather additional facts from the blood relations of the patient by asking relevant questions. By obtaining information from family members, MDCA system 250 can generate a more comprehensive understanding of the medical situation.

To accomplish this, the MDCA system 250 employs a sophisticated algorithm (e.g., decision trees, random forest, support vector machines, naïve Bayes, neural networks, etc.) that analyzes the collected data and generates a response maturity score and level of derivation indicator. These indicators help assess the reliability and relevance of the information provided by the blood relations. The response maturity score evaluates the completeness and accuracy of the responses, while the level of derivation indicator determines the extent to which the information obtained from the blood relations contributes to the overall symptom analysis.

By incorporating these indicators, the MDCA system 250 enhances its capability to assess the reliability of the gathered information and effectively integrate it into the symptom analysis process. This ensures that the system provides accurate and dependable results, benefiting both medical professionals and patients in making informed decisions regarding medical conditions and treatments. Additional questions can be generated when the collected information is deemed to insufficiently contribute to the overall symptom analysis. For example, the maturity score may be below a predefined score threshold and/or the level of derivation indicator may exceed a predefined derivation threshold.

The use of a questionnaire is exemplary only and not intended to be limiting. Other means of collecting medical information can be used. For example, the medical professional may use questionnaires, forms, online platforms, oral interviews, and/or the like to help collect, organize, and record the family medical history accurately.

Referring back to FIG. 2, the NLU engine 260 is designed to receive patient medical data 202 and family data 204. The patient medical data 202 consists of various symptoms identified and provided by the medical professional, as well as other patient information detailed above. Family data 204 includes information about different family members in the family tree, such as that which has been detailed above. To improve its understanding capabilities, the NLU engine 260 creates a knowledge corpus 210 of associated terms by analyzing the information about family members in the family tree. This analysis helps establish connections between symptoms and disease traits. Understanding genetics requires considering family member relationships and connections. By identifying links between immediate and distant relatives through genealogical charts (e.g., family tree), it becomes easier to identify patterns in inheritable traits.

It should be noted that communication styles among family members can vary significantly based on factors such as culture, education level, social background, and age group. The information about family members can include their relationships with each other and sample sentences of various genres, such as direct, alluding, cryptic, sensitive, and narrative. Additionally, the family member information may encompass alternate languages, dialects, or tribal references, thereby accommodating diverse linguistic expressions. The provided answers relating to the medical history of each family member offer a medical narration.

Noisy content removal component 262 is configured to filter the verbatim medical narrations about the medical conditions and disease progression faced by the different impacted family members. Medical narration refers to the process of verbally or non-verbally describing the medical conditions, situations, and disease progression faced by different individuals within a family. This narration includes a variety of information such as symptoms, treatment experiences, and the impact of the disease on the affected individuals. It may involve different genres of language, including direct descriptions, alluding references, cryptic statements, sensitive discussions, and narrative accounts. The purpose of medical narration is to provide a comprehensive understanding of the medical history and genetic traits within a family.

Noisy content removal component 262 is responsible for removing any irrelevant or unnecessary information from the narrations, ensuring that only the relevant and essential details are retained. The noisy content removal component 262 can use Natural Language Processing (NLP) and Machine Learning (ML) techniques to distinguish noise from other data. It can employ statistical analysis, rule-based filtering, machine learning models, and/or language models to identify patterns or characteristics of noise in the data. By removing the noise from the narration, noisy content removal component 262 helps in providing a crisp and concise version of the information, which can then be further processed and analyzed by NLU engine 260. This ensures that NLU engine 260 receives accurate and relevant data, improving its ability to understand and analyze the symptoms and disease traits linked to the family members.

NLU engine 260 receives and analyzes the filtered content, extracting valuable insights and understanding the context and meaning behind the words. NLU engine 260 can utilize methods such as named entity recognition, sentiment analysis, topic modeling, relationship extraction, contextual understanding, and/or knowledge graphs to perform this task. This analysis is of utmost importance as it enables healthcare professionals to comprehend and make sense of the medical conditions, disease progression, and genetic traits discussed by various family members. One of the key aspects of the analysis is the examination of the patient's family's medical history. By delving into this historical data, NLU engine 260 identifies any potential patterns of inheritance, genetic commonalities, and disease trails that may exist within the family. This comprehensive analysis of the medical history provides crucial information that can aid in predicting disease likelihood, understanding genetic predispositions, and evaluating potential risk factors. Once NLU engine 260 completes its analysis, the output is utilized by tracer component 264.

Tracer component 264 is configured to receive the output to establish a linked tree of related words. This linked tree serves as a representation that enhances the understanding and analysis of medical conditions and disease progression. By connecting related words, the linked tree enables a comprehensive analysis of the information, allowing for a deeper exploration of the relationships and connections between different terms. Establishing a linked tree of related words also helps in identifying key factors and patterns within the medical narrative. By mapping out these connections, it becomes easier to identify common symptoms, genetic traits, or patterns of disease inheritance within the family. This representation helps in conveying complex medical information in a clear and concise manner, fostering better communication and promoting a holistic approach to understanding and addressing medical conditions and disease progression.

Inheritance predictor component 266 is configured to, using the linked tree of related words, determine likelihood paths of a medical cause through the family based on symptoms extracted from the medical narrations. “Medical cause” refers to the underlying reason for a particular disease or health issue. In other words, it explains why someone may experience certain symptoms or signs that indicate an illness. Medical causes can include genetic factors, environmental triggers, lifestyle choices, or exposure to harmful substances. Identifying the root cause of an illness is important because it allows medical professionals to develop effective treatments and prevention strategies. By analyzing the symptoms extracted from the narrations provided by the family, inheritance predictor component 266 employs advanced algorithms and techniques to identify patterns and connections within the family tree. These patterns and connections are then used to predict the probability of certain traits or conditions being passed down from one generation to another. In doing so, inheritance predictor component 266 significantly enhances the accuracy and reliability of the symptom analysis process.

The utilization of the linked tree structure is particularly advantageous for inheritance predictor component 266. This structure allows for the representation of complex family relationships and enables the component to consider various factors such as direct and indirect inheritance, genetic predispositions, and even potential carriers of certain conditions within the family. Inheritance predictor component 266 not only contributes to the accuracy of the symptom analysis process but also aids in identifying potential hereditary risks and providing valuable information to patients regarding their family's health history. This predictive capability can assist healthcare professionals in making more informed decisions and recommending appropriate preventive measures or treatment options.

Characterizer component 268 is configured to match these paths with the requirements of different clinical trials, indicating potential intersections and eligibility based on symptom matching. To achieve accurate symptom analysis, characterizer component 268 utilizes linguistic equivalences to match symptoms. It derives these equivalences and ensures that the symptom analysis is precise. To make clinical trial opportunities more accessible to potential patients, characterizer component 268 further utilizes natural language processing techniques to analyze patient data. This analysis involves detecting patterns in verb usage and sentence structure to identify relevant information such as the presence and severity of symptoms, medical history, and other health factors. By comparing the collected patient data with the requirements of each clinical trial, a matrix is generated. This matrix displays eligible studies and potential enrollees, which can assist participants in making informed decisions during a consent process.

The MDCA system 250 comprises various components and functions that utilize language grammar, description abstraction, and strength expression to process complex symptom data. These methods enhance the system's ability to understand and analyze medical situations. Moreover, the system is designed to accommodate different languages used by family members and caregivers when describing medical conditions. It incorporates synonyms and variations of words and symptoms, thereby improving its adaptability and accuracy in symptom analysis.

The MDCA system 250 enables the determination of symptoms based on information provided by the patient's blood relations. By analyzing the “facts” expressed by these relatives, it can identify relevant symptoms that may be indicative of a medical condition. Also, the system establishes connections between the expressed symptoms and potential symptoms provided by medical professionals. This allows for a comprehensive understanding of the symptoms and aids in accurate diagnosis and treatment planning.

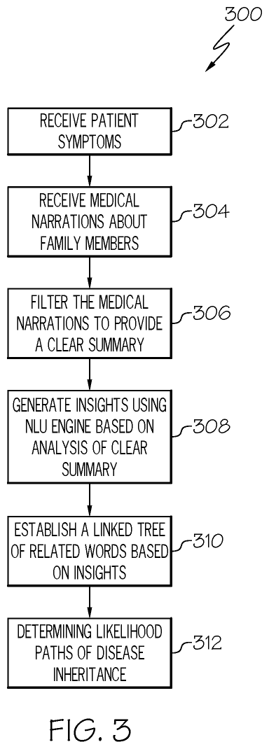

FIG. 3 depicts a flowchart diagram related to determining a genetic predisposition of a medical cause from a family tree analysis. Referring additionally to FIG. 2, at 302, the approach involves receiving medical symptoms (e.g., as part of patient medical data 202) of a patient. At 304, medical narrations about family members (e.g., as part of family data 204), which are organized in a family tree, are also received. At 306, noisy content removal component 262 filters the narrations to provide a summary of relevant information. At 308, NLU engine 260 generates insights based on an analysis of the summary. Subsequently, at 310, tracer component 264 establishes a linked tree of related words based on the derived insights. At 312, inheritance predictor component 266 determines likelihood paths of a medical cause through the family using the linked tree of related words and symptoms obtained from the medical narrations. An intersection of the paths with requirements to participate in different clinical trials can then be shown by matching the symptoms, directly or derived by linguistic equivalences.

The illustrated steps in FIG. 3 are not necessarily performed in the order shown, and some steps may be performed concurrently or in a different order than shown. The flowchart diagram is intended to illustrate the general flow of the method and is not intended to be limiting. Additional steps may be added, or some steps may be omitted without departing from the scope of the invention. The steps may be performed by a computer program or by a combination of hardware and software. The flowchart diagram may be implemented using any suitable programming language or tool.

The disclosed approach offers several advantages over current methods. It enables patients to easily identify clinical trials suitable for their symptoms and family medical history. Additionally, it assists medical professionals in establishing the likely inheritance pattern associated with a disease by providing information about affected family members' medical history. For established genetic diseases, the approach ensures that family members receive alerts about the disease, including its cause, symptoms, and available treatment options. This proactive approach allows families to assess their risks and potentially take preventive measures. In addition, the approach has the capability to detect and map behavioral or seasonal traits that may resemble symptoms but follow a different pathway. This enhances the ability to accurately analyze and interpret patient data, providing a comprehensive understanding of her condition. The approach introduces a sophisticated system that utilizes natural language analytics and understanding techniques to analyze patient symptoms and family history and provide a comprehensive and accurate understanding of the patient's condition. It accommodates multiple languages, diverse use cases, and enables the identification of clinical trial eligibility and inheritance patterns. The ability to filter noise, establish connections between terms, and match symptoms enhances the efficiency and effectiveness of medical professionals in diagnosing and treating patients.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for determining a genetic predisposition of a medical cause from a family tree analysis, comprising:
receiving, by a natural language understanding engine, medical symptoms of a patient;
generating, by the natural language understanding engine, a set of questions from a supporting symptoms list;
receiving, by the natural language understanding engine, medical narrations from family members of the patient represented in a family tree by asking the family members the generated set of questions;
filtering, by a cognitive system, the medical narrations to provide a summary;
generating, by the natural language understanding engine, insights based on the summary using a natural language processing technique;
establishing, by the cognitive system, a linked tree of related words based on the insights;
determining, by the cognitive system, likelihood paths through the family based on symptoms identified in the linked tree of related words; and
matching, by the cognitive system, the determined likelihood paths with a known inheritance pattern of a disease to provide an alert to the family members about the disease.

2. The method of claim 1, wherein the insights include at least one of an inheritance pattern, genetic commonality, or disease trail.

3. The method of claim 1, further comprising analyzing, by natural language understanding engine, the answers to the generated set of questions to establish a response maturity score and level of derivation indicator.

4. The method of claim 3, further comprising generating, by the cognitive system, a second set of questions based on at least one of the response maturity score or level of derivation indicator.

5. The method of claim 1, further comprising analyzing, by the natural language understanding engine, the answers to the set of generated questions to determine a set of symptoms.

6. The method of claim 1, further comprising matching, by the cognitive system, the determined likelihood paths with a set of requirements of a clinical trial to identify an intersection.

7. The method of claim 6, further comprising assessing, by the cognitive system, eligibility of the patient in the clinical trial based on the intersection.

8. A computing system for determining a genetic predisposition of a medical cause from a family tree analysis, comprising:
a processor;
a memory device coupled to the processor; and
a computer readable storage device coupled to the processor, wherein the storage device contains program code executable by the processor via the memory device to implement a method, the method comprising:
receiving, by a natural language understanding engine, medical symptoms of a patient;
generating, by the natural language understanding engine, a set of questions from a supporting symptoms list;
receiving, by the natural language understanding engine, medical narrations from family members of the patient represented in a family tree by asking the family members the generated set of questions;
filtering, by a cognitive system, the medical narrations to provide a summary;
generating, by the natural language understanding engine, insights based on the summary using a natural language processing technique;
establishing, by the cognitive system, a linked tree of related words based on the insights;
determining, by the cognitive system, likelihood paths through the family based on symptoms identified in the linked tree of related words; and
matching, by the cognitive system, the determined likelihood paths with a known inheritance pattern of a disease to provide an alert to the family members about the disease.

9. The computing system of claim 8, wherein the insights include at least one of an inheritance pattern, genetic commonality, or disease trail.

10. The computing system of claim 8, the method further comprising analyzing, by the natural language understanding engine, the answers to the generated set of questions to establish a response maturity score and level of derivation indicator.

11. The computing system of claim 10, the method further comprising generating, by the natural language understanding engine, a second set of questions based on at least one of the response maturity score or level of derivation indicator.

12. The computing system of claim 8, the method further comprising analyzing, by the natural language understanding engine, the answers to the set of generated questions to determine a set of symptoms.

13. The computing system of claim 8, the method further comprising matching, by the cognitive system, the determined likelihood paths with a set of requirements of a clinical trial to identify an intersection.

14. The computing system of claim 13, the method further comprising assessing, by the cognitive system, eligibility of the patient in the clinical trial based on the intersection.

15. A computer program product for determining a genetic predisposition of a medical cause from a family tree analysis, the computer program product comprising a computer readable storage device, and program instructions stored on the computer readable storage device, to:
receive, by a natural language understanding engine, medical symptoms of a patient;
generate, by the natural language understanding engine, a set of questions from a supporting symptoms list;
receive, by the natural language understanding engine, medical narrations from family members of the patient represented in a family tree by asking the family members the generated set of questions;
filter, by a cognitive system, the medical narrations to provide a summary;
generate, by the natural language understanding engine, insights based on the summary using a natural language processing technique;
establish, by the cognitive system, a linked tree of related words based on the insights;
determine, by the cognitive system, likelihood paths through the family based on symptoms identified in the linked tree of related words; and
match, by the cognitive system, the determined likelihood paths with a known inheritance pattern of a disease to provide an alert to the family members about the disease.

16. The computer program product of claim 15, wherein the insights include at least one of an inheritance pattern, genetic commonality, or disease trail.

17. The computer program product of claim 15, further comprising program instructions stored on the computer readable storage device to analyze, by the natural language understanding engine, the answers to the generated set of questions to establish a response maturity score and level of derivation indicator.

18. The computer program product of claim 17, further comprising program instructions stored on the computer readable storage device to generate, by the natural language understanding engine, a second set of questions based on at least one of the response maturity score or level of derivation indicator.

19. The computer program product of claim 15, further comprising program instructions stored on the computer readable storage device to analyze, by the natural language understanding engine, the answers to the set of generated questions to determine a set of symptoms.

20. The computer program product of claim 15, further comprising program instructions stored on the computer readable storage device to match, by the cognitive system, the determined likelihood paths with a set of requirements of a clinical trial to identify an intersection.
